(19) United States
(12) Patent Application Publication
(10) Pub. No.: US 2004/0102751 A1
(43) Pub. Date: May 27, 2004

(54) Absorbent article with reinforced absorbent structure

(75) Inventor: Kenneth R. Schueler JR., Appleton, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/306,074

(22) Filed: Nov. 27, 2002

(51) Int. Cl. A61F 13/15; A61F 13/20

(52) U.S. Cl. 604/367

(57) ABSTRACT

An absorbent structure can include a matrix of fibers, wherein the matrix is reinforced with a reinforcing member which strengthens the fibrous matrix against tearing, cracking and bunching in manufacture and/or use. In addition, the reinforcing member is shaped to provide resistance to permanent deformation in the thickness of the fibrous matrix. The reinforcing member has a three dimensional configuration which provides resistance to thickness deformation. Thus, the fibrous matrix is better able to retain its liquid permeability and capacity in use.

### BACKGROUND OF THE INVENTION

This invention generally relates to an absorbent article and a reinforced absorbent structure for such an article. The absorbent structure may include absorbent fibers in a matrix and a reinforcing member embedded within the fibrous matrix. The reinforcing member is shaped to stabilize the fibrous matrix at least in the thickness direction. The reinforced absorbent structure can be employed in absorbent articles, such as disposable diapers, child's training pants, feminine care articles, incontinence articles, bandages, and the like.

Absorbent articles, such as for disposable absorbent garments, may include absorbent structures or cores conventionally formed by air forming or air laying techniques. For example, the manufacture of the absorbent core may begin by fiberizing a fibrous sheet of cellulosic or other suitable absorbent material in a conventional fiberizer, or other shredding or comminuting device, to form discrete fibers. In addition, particles of superabsorbent material are mixed with the discrete fibers. The fibers and superabsorbent particles are then entrained in an air stream and directed to a foraminous forming surface upon which the fibers and superabsorbent particles are deposited to form an absorbent fibrous web. In addition, bonding agents or other strengthening components may be incorporated to provide a more stabilized web.

Other techniques have also been employed to form webs of stabilized absorbent material. Such techniques have included dry-forming techniques, wet-laying techniques, foam-forming techniques, and various wet-forming techniques. The resulting webs of absorbent material have included absorbent fibers, natural fibers, synthetic fibers, superabsorbent materials, binders, and strengthening components in desired combinations. However formed, the absorbent web may then be stored or immediately directed for further processing (e.g., being cut into individual absorbent cores) and assembly with other components to produce a final absorbent article.

Integrity of an absorbent core formed from such an absorbent material web is desirable to avoid bunching, clumping, cracking and separating of the absorbent core in either a wet or a dry state. This improves the fit and comfort to the wearer of an absorbent article incorporating the absorbent core even as the article receives insults. Sagging and drooping of the absorbent article can cause gaps between the article and the wearer's body which may lead to leaking. It is known that stability of the absorbent core in the thickness direction of the core maintains or increases the permeability of the core to liquid and the intake capacity of the core. Preferably, stability in the thickness direction should be maintained in both wet and dry conditions of the absorbent core.

Absorbent material webs have been strengthened by adding reinforcing members on at least one side of the absorbent material webs. Such reinforcing members have included reinforcement filaments, tissue layers, fabric layers and netting materials. Generally speaking, reinforcing members of this type do not significantly augment the stability of the absorbent material in the thickness direction. It is also known to add staple binder fibers to the absorbent materials upon formation of the absorbent material web. The binder fibers are activated by heat to produce adhesion of the fibers and other absorbent materials in the web. The use of binder fibers can increase the thickness direction stability of an absorbent core formed from the absorbent material web, but requires a considerably more expensive manufacturing process.

### SUMMARY OF THE INVENTION

An absorbent article constructed accordingly to the principles of the present invention can include a matrix of fibers, wherein the fibrous matrix is reinforced with a reinforcing member. The reinforcing member is connected to the fibrous matrix by connections which occur under certain controlled conditions in the manufacture of the absorbent core. No additional securing steps are required. In another aspect, the reinforcing member can be shaped to provide additional reinforcement against collapse of the fibrous matrix. In a further aspect, the reinforcing member may include multiple layers of material which may be shaped in the same way or differently.

In one aspect of the present invention, an absorbent structure for absorbing liquid comprises an absorbent member being at least partially made of fibers and having a thickness, and a reinforcing member at least partially embedded in the absorbent member for maintaining the structural integrity of the absorbent member. The reinforcing member is constructed and arranged relative to the absorbent member to resist compaction of the absorbent member at least in the thickness direction of the absorbent member.

In another aspect of the invention, an absorbent structure for absorbing liquid comprising an absorbent member as set forth in the preceding paragraph, and a reinforcing member at least partially embedded in the absorbent member for maintaining the structural integrity of the absorbent member. The reinforcing member being attached to fibers from the absorbent member, the reinforcing member having a median surface when the absorbent member is laid flat, the reinforcing member extending on both sides of said median surface.

Other features of the invention will be in part apparent and in part pointed out hereinafter.

### BRIEF DESCRIPTION OF THE DRAWINGS

FIG. 1 is a top plan view of an absorbent article with parts broken away to show internal construction;

FIG. 2 is a schematic, exploded view, in cross-section, of an absorbent article that incorporates the present invention;

FIG. 3 is longitudinal section of and absorbent core of the absorbent article showing a scrim reinforcing member having an accordion configuration in which folds extend lengthwise of the core;

FIG. 3A is an enlarged fragment of the section of FIG. 3 illustrating a median plane of the scrim reinforcing member;

FIG. 3B is a cross section of an absorbent core showing a scrim reinforcing member having an accordion configuration in which folds extend widthwise of the core;

FIG. 4 is a longitudinal section of an absorbent core showing another arrangement of a scrim reinforcing member in two layers;

FIG. 5 is a transverse section of an absorbent core showing still another arrangement of a scrim reinforcing member projecting from the absorbent core;

FIG. 6 is a transverse section of an absorbent core in two portions, showing a further arrangement of a scrim reinforcing member extending between and spacing the absorbent core portions;

FIG. 7 is a perspective of a three dimensional box lattice reinforcing member;

FIG. 8 is an enlarged, fragmentary side elevation of the box lattice in a collapsed configuration;

FIG. 9 is an enlarged, fragmentary side elevation of a box spring reinforcing member;

FIG. 10 is a perspective of the box spring reinforcing member;

FIG. 11 is a perspective of square tubes of reinforcing material;

FIG. 12 is a perspective of double helix reinforcing members;

FIG. 13 is a perspective of single strand reinforcing members;

FIG. 14 is a schematic elevation of an air forming machine for forming absorbent cores;

FIG. 15 is a schematic perspective of a forming drum of the air forming machine of FIG. 14; and

FIG. 16 is a longitudinal section, in perspective of a form member of the forming drum.

Corresponding reference characters indicate corresponding parts throughout the several views of the drawings.

### DETAILED DESCRIPTION OF THE DRAWINGS

The present invention has application in various types of desired absorbent articles. Such articles can include, for example, infant diapers, children's training pants, feminine care articles, adult incontinence garments, bandages and the like for use in absorbing various body exudates. The articles may be, but are not necessarily, disposable and intended for limited use.

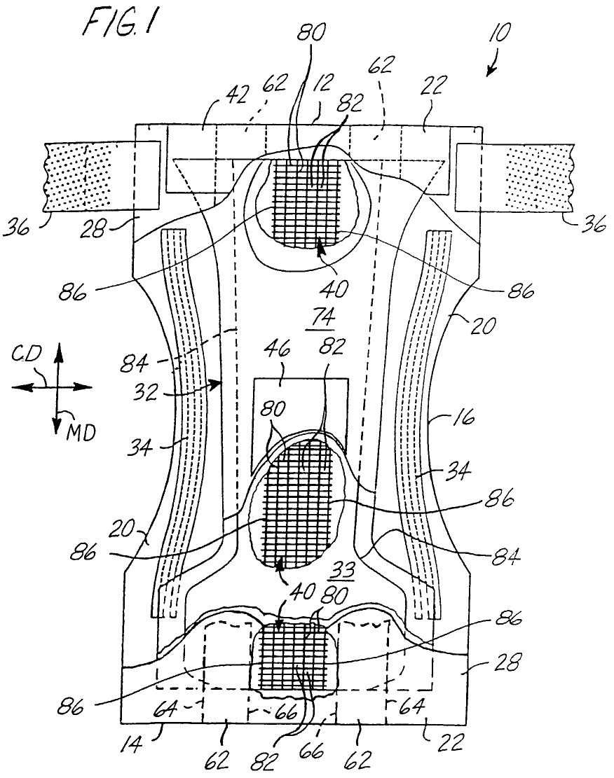

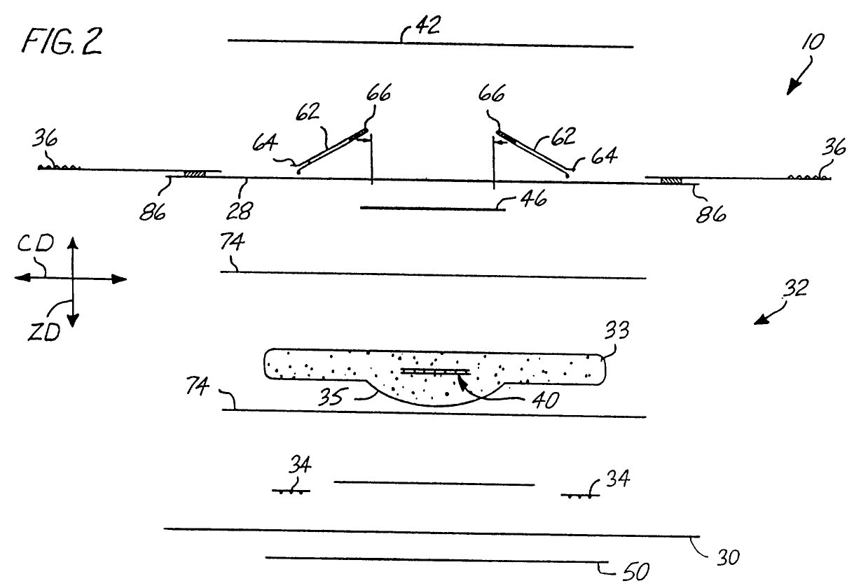

Referring now to the drawings, and in particular to FIGS. 1 and 2, an absorbent article constructed according to the principles of the present invention is shown in the form of a diaper 10 unfolded and laid flat with substantially all elastic induced gathering and contraction removed. The diaper 10 extends lengthwise in a longitudinal or machine-direction MD, widthwise in a lateral or cross-direction CD, and has a thickness in a “z” or thickness direction ZD. For the purposes of the present disclosure, the machine-direction MD lies generally parallel to the plane of the diaper 10, and extends generally along a line that lies between opposed end regions of the diaper. The cross-direction CD lies generally parallel to the plane of the article, and is aligned perpendicular to the longitudinal-direction MD. The z-direction ZD is aligned substantially perpendicular to both the longitudinal-direction MD and the cross-direction CD, and extends through the thickness of the diaper 10. In FIG. 1, the bodyside surface of the diaper which contacts the wearer is facing the viewer and, portions of the structure are partially cut away to more clearly show the interior construction of the diaper article 10. The outer edges of the diaper define a periphery with longitudinally extending side edge margins 20 and laterally extending end edge margins 22. The side edges define leg openings for the diaper 10.

With regard to the designated surfaces of the article, the various inward or bodyside surfaces are configured to face toward the body of the wearer when the article is placed about the wearer. The designated outward surfaces of the article are configured to face away from the wearer's body when the article is placed about the wearer. The diaper 10 may have any desired shape, such as rectangular, I-shaped, a generally hourglass shape, or a T-shape. With the T-shape, the crossbar of the “T” may comprise the front waistband portion of the diaper, or may alternatively comprise the rear waistband portion of the diaper.

The diaper 10 includes an absorbent structure, generally indicated at 32, having an absorbent core 33 (broadly, “an absorbent member”) which includes absorbent fibers and superabsorbent material (SAM). The absorbent core 33 may also include other fibers which are not absorbent. A web of scrim 40 (broadly, “a reinforcing member”) is located roughly in the middle of the absorbent core 33 for reinforcing the fibrous absorbent core to enhance the integrity of the core under loads as will be described more fully hereinafter. The actual position of the scrim between major surfaces of the core 33 varies over the core because in the illustrated embodiment, the core may have a non-constant thickness. However, the reinforcing member of the present invention may lessen the need for thickness and/or basis weight variations within an absorbent core. It is to be understood that the scrim can be placed away from the middle, toward one side or the other within the absorbent core thickness and still fall within the scope of the invention. A backsheet layer 30 and a liquid permeable topsheet layer 28 are arranged opposite each other and the absorbent structure 32 is located between the backsheet layer and topsheet layer. Typically, the backsheet layer 30 is liquid impermeable, but may be liquid permeable without departing from the scope of the present invention. The illustrated diaper 10 has a first or back waistband portion 12, a second or front waistband portion 14 and an intermediate or crotch portion 16 that interconnects the back and front waistband portions. In use, the diaper 10 is fitted onto the lower torso and around the upper legs of a wearer (e.g., a child or infant), assuming a curved, three dimensional configuration in which parts of the back and front waistbands portions 12, 14 overlie or lie in close proximity to each other.

A fastening system including fastener tabs 36 and a landing zone patch 50 for receiving the fastener tabs to interconnect the back waistband portion 12 with the front waistband portion 14 to hold the article on a wearer so that the back portion overlaps the front portion. However, a fastening system (not shown) could be used in which a front waistband portion overlaps the back waistband portion. In such optional arrangements, the front waistband portion would be the “first” waistband portion and the back waistband region would be the “second” waistband portion. The diaper also has a system of elastomeric gathering members, including leg elastics 34 to draw the diaper 10 around the legs and a waist elastic 42 (located in the back waistband portion 12) to draw the diaper around the waist.

The backsheet layer 30 is located along an outside surface of the absorbent structure 32 and may be composed of a liquid permeable material, but desirably comprises a material which is configured to be substantially impermeable to liquids. For example, a typical backsheet layer 30 can be manufactured from a thin plastic film, or other flexible, substantially liquid-impermeable material. As used in the present disclosure, the term “flexible” refers to materials which are compliant and which will readily conform to the general shape and contours of the wearer's body. The backsheet layer 30 can prevent the exudates contained in absorbent structure 32 from wetting articles, such as bedsheets and overgarments, which contact diaper 10. In particular embodiments of the invention, backsheet layer 30 can include a film, such as a polyethylene film, having a thickness of from about 0.012 millimeters (0.5 mil) to about 0.051 millimeters (2.0 mil). For example, the backsheet film can have a thickness of about 0.032 millimeters (1.25 mil).

Alternative constructions of the backsheet layer 30 may comprise a woven or non-woven fibrous web which has been totally or partially constructed or treated to impart the desired levels of liquid impermeability to selected regions that are adjacent or proximate the absorbent structure. For example, the backsheet layer 30 may include a gas-permeable, nonwoven fabric material laminated to a facing surface of a polymer film material that may or may not be gas-permeable. Ordinarily, the fabric material is attached to an outward-facing surface of the polymer film material. Other examples of fibrous, cloth-like backsheet layer materials are a stretch-thinned or a stretch-thermal-laminate material composed of a 0.015 mm (0.6 mil) thick polypropylene blown film and a 23.8 g/m² (0.7 osy) polypropylene spunbond material (2 denier fibers).

In particular arrangements, a substantially liquid impermeable, vapor permeable backsheet layer 30 may be a composite material which includes a vapor permeable film adhesively laminated to a spunbond material. The vapor permeable film can be obtained from Exxon Chemical Products Incorporated, under the tradename EXXAIRE. The film can include 48-60 weight percent (wt %) linear low density polyethylene and 38-50 wt % calcium carbonate particulates that may be uniformly dispersed and extruded into the film. The stretched film can have a thickness of about 0.018 mm (0.7 mil) and a basis weight of 16-22 g/m². The spunbond material can be adhesively laminated to the film, and can have a basis weight of about 27 g/m². The spunbond material can be made using conventional spunbond technology, and can include filaments of polypropylene having a fiber denier of 1.5-3 dpf. The vapor-permeable film may be adhered to the spunbond material using a pressure sensitive, hot melt adhesive at an add-on rate of about 1.6 g/m², and the adhesive can be deposited in the form of a pattern of adhesive swirls or a random fine fiber spray. Another example of a suitable microporous film can be a PMP-1 material, which is available from Mitsui Toatsu Chemicals, Inc., a company having offices in Tokyo, Japan; or an XKO-8044 polyolefin film available from 3M Company of Minneapolis, Minn.

The liquid impermeable, vapor permeable backsheet layer 30 may alternatively include a highly breathable stretch thermal laminate material (HBSTL). The HBSTL material can include a polypropylene spunbond material thermally attached to a stretched breathable film. For example, the HBSTL material may include a 20.4 g/m² (0.6 osy) polypropylene spunbond material thermally attached to an 18.7 g/m² stretched breathable film. The breathable film may include two skin layers with each skin layer composed of 1-3 wt % EVA/catalloy. The breathable film may also include 55-60 wt % calcium carbonate particulates, linear low density polyethylene, and up to 4.8% low density polyethylene. The stretched breathable film can include a thickness of 0.011-0.013 mm (0.45-0.50 mils) and a basis weight of 18.7 g/m². The spunbond material can be thermally bonded to the breathable film, and can have a basis weight of about 20.4 g/m². The spunbond material can have a fiber denier of 1.5-3 dpf, and the stretched breathable film can be thermally attached to the spunbond material using a “C-star” pattern that provides an overall bond area of 15-20%.

The various types of such materials have been employed to form the backsheet layer or outer cover of disposable diapers, such as HUGGIES disposable diapers which are commercially available from Kimberly-Clark Corporation. Optionally, however, the article may include a separate component that is additional to the backsheet layer. The backsheet layer 30 may also be embossed or otherwise provided with a pattern or matte finish to exhibit a more aesthetically pleasing appearance.

The topsheet layer 28 presents a body-facing surface that is compliant, soft-feeling, and non-irritating to the wearer's skin. Further, the topsheet layer 28 can be less hydrophilic than absorbent structure 32, and is sufficiently porous to be liquid permeable, permitting liquid to readily penetrate through its thickness to reach the absorbent structure. A suitable topsheet layer 28 may be manufactured from a wide selection of materials, such as porous foams, reticulated foams, apertured plastic films, natural fibers (for example, wood or cotton fibers), synthetic fibers (for example, polyester or polypropylene fibers), or a combination of natural and synthetic fibers. The topsheet layer 28 is typically employed to help isolate the wearer's skin from liquids held in absorbent structure 32. Various woven and nonwoven fabrics can be used for topsheet layer 28. For example, the topsheet layer may be composed of a meltblown or spunbonded web of the desired fibers, and may also be a bonded-carded-web. The various fabrics can be composed of natural fibers, synthetic fibers or combinations thereof. For the purposes of the present description, the term “nonwoven web” means a web of fibrous material that is formed without the aid of a textile weaving or knitting process. The term “fabrics” is used to refer to all of the woven, knitted and nonwoven fibrous webs.

The topsheet layer fabrics may be composed of a substantially hydrophobic material, and the hydrophobic material may optionally be treated with a surfactant or otherwise processed to impart a desired level of wettability and hydrophilicity. In a particular embodiment of the invention, topsheet layer 28 is a nonwoven, spunbond polypropylene fabric composed of about 2.8-3.2 denier fibers formed into a web having a basis weight of about 22 gsm and density of about 0.06 gm/cc. The fabric can be surface treated with an operative amount of surfactant, such as about 0.28% TRITON X-102 surfactant. Other types and amounts of operative surfactants may alternatively be employed. The surfactant can be applied by any conventional means, such as spraying, printing, brush coating or the like.

The topsheet layer 28 and backsheet layer 30 are connected or otherwise associated together in a suitable manner. As used herein, the term “associated” encompasses configurations in which topsheet layer 28 is directly joined to backsheet layer 30 by affixing topsheet layer directly to backsheet layer, and configurations wherein topsheet layer is indirectly joined to backsheet layer by affixing topsheet layer to intermediate members which in turn are affixed to backsheet layer. The topsheet layer 28 and backsheet layer 30 can, for example, be joined to each other in at least a portion of the diaper periphery in a suitable manner such as by adhesive bonding, sonic bonding, thermal bonding, pinning, stitching or any other attachment technique known in the art, as well as combinations thereof. For example, a uniform continuous layer of adhesive, a patterned layer of adhesive, a sprayed pattern of adhesive or an array of separate lines, swirls or spots of construction adhesive may be used to affix the topsheet layer to the backsheet layer. It should be readily appreciated that the above-described attachment mechanisms may also be employed to suitably interconnect, assemble and/or affix together the various other component parts of the articles that are described herein.

The diaper 10 also includes a surge management member 46 which helps to decelerate and diffuse surges or gushes of liquid that may be rapidly introduced into the absorbent structure of the article. Desirably, the surge management member 46 can rapidly accept and temporarily hold the liquid prior to releasing the liquid into the absorbent structure 32. In the illustrated embodiment, for example, the surge member 46 is located on an inwardly facing body side surface of the topsheet layer 28. Alternatively, the surge member 46 may be located adjacent to an outer side surface of the topsheet layer 28. Accordingly, the surge member 46 is interposed between the topsheet layer 28 and the absorbent structure 32. Examples of suitable surge management members 46 are described in U.S. Pat. No. 5,486,166 entitled FIBROUS NONWOVEN WEB SURGE LAYER FOR PERSONAL CARE ABSORBENT ARTICLES AND THE LIKE by C. Ellis and D. Bishop, which issued Jan. 23, 1996; and U.S. Pat. No. 5,490,846 entitled IMPROVED SURGE MANAGEMENT FIBROUS NONWOVEN WEB FOR PERSONAL CARE ABSORBENT ARTICLES AND THE LIKE by C. Ellis and R. Everett, which issued Feb. 13, 1996; the entire disclosures of which are hereby incorporated by reference. However, it is to be understood that the surge management member 46 can be omitted without departing from the scope of the present invention.

Elasticized containment flaps 62 extend generally lengthwise in the machine-direction MD of the diaper 10. The containment flaps 62 are typically positioned laterally inboard from the leg elastics 34, and substantially symmetrically placed on each side of the longitudinal centerline of the diaper. In the illustrated arrangements, each containment flap 62 has a substantially fixed edge portion 64 and a substantially moveable edge portion 66, and is operably elasticized to help each containment flap to closely contact and conform to the contours of the wearer's body. Examples of suitable containment flap constructions are described in U.S. Pat. No. 4,704,116 entitled DIAPERS WITH ELASTICIZED SIDE POCKETS by K. Enloe which issued Nov. 3, 1987, the entire disclosure of which is hereby incorporated by reference. The containment flaps 62 may be composed of a wettable or a non-wettable material, as desired. In addition, the containment flap material may be substantially liquid-impermeable, may be permeable to only gas or may be permeable to both gas and liquid. The level of permeability of the containment flap material may be substantially the same as or different than the permeability of other components of the article. Other suitable containment flap configurations are described in U.S. Pat. No. 5,562,650 entitled ABSORBENT ARTICLE HAVING AN IMPROVED SURGE MANAGEMENT by R. Everett et al., which issued Feb. 13, 1996, the disclosure of which is hereby incorporated by reference.

In optional, alternative configurations of the invention, diaper 10 may include internal, elasticized, containment waist flaps, such as those described in U.S. Pat. No. 4,753,646 entitled DIAPER WITH WAIST FLAPS by K. Enloe which issued Jun. 28, 1988; and in U.S. Pat. No. 5,904,675 entitled AN ABSORBENT ARTICLE WITH IMPROVED ELASTIC MARGINS AND CONTAINMENT SYSTEM by D. Laux et al., which issued May 18, 1999; the entire disclosures of which are hereby incorporated by reference. Similar to the construction of the containment flaps 62, the containment waist flaps may be composed of a wettable or non-wettable material, as desired. The waist flap material may be substantially liquid-impermeable, permeable to only gas, or permeable to both gas and liquid.

The landing zone patch 50 provides a target area for releasable and re-attachable securement with the fastener tabs 36. The landing member patch 50 is positioned on the front waistband portion 14 of the diaper 10 and located on the outward surface of the backsheet layer 30 in the illustrated embodiment. Alternatively, the landing member patch 50 could be positioned on an inward surface of the diaper 10, such as the bodyside surface of the topsheet layer 28, or at any other suitable location. Particular arrangements of the invention can include one or more landing members that can be directly or indirectly attached to the second waistband portion 14. The landing member 50 can be composed of a substantially non-elastomeric material, such as polymer films or tapes, woven fabrics, nonwoven fabrics or the like, as well as combinations thereof. The landing member 50 could also be made of a substantially elastomeric material, such as a stretch-bonded-laminate (SBL) material, an elastomeric neck-bonded-laminate (NBL) material, an elastomeric film, an elastomeric foam material, or the like, which is elastomerically stretchable at least along the lateral direction CD.

The fastener tabs 36 are located at rearward portions of the side edges 20 near the back waistband portion 12, but could be located at front portions of the side edges near the front waistband portion 14. The fastener tab 36 can be made of a substantially non-elastomeric material, such as polymer films or tapes, woven fabrics, nonwoven fabrics or the like, as well as combinations thereof. Optionally, the fastener tabs 36 may be composed of a substantially elastomeric material, such as a stretch-bonded-laminate (SBL) material, a neck-bonded-laminate (NBL) material, an elastomeric film, an elastomeric foam material, or the like, which is elastomerically stretchable at least in the lateral direction CD.

In the various aspects and configurations of the invention, the fastening mechanism between the selected first fastener component and the selected, second fastener component may be adhesive, cohesive, mechanical or combinations thereof. In the context of the present invention, a mechanical fastening system is a system that includes cooperating, first and second components that mechanically interengage to provide a desired securement. Desirably, the first and second fastener components include complementary elements of a cooperatively interengaging mechanical fastening system. The mechanical fastener components can be provided by mechanical-type fasteners such as hooks, buckles, snaps, buttons and the like, which include cooperating and complementary, mechanically interlocking components.

As shown in the illustrated embodiment, the mechanical fastening system is of the hook-and-loop type. Such fastening systems typically include attachment members having the form of a “hook” or hook-like, male component, and include a cooperating “loop” or loop-like, female component that engages and releasably interconnects with the hook component. Desirably, the interconnection is selectively releasable and re-attachable. Conventional systems are, for example, available under the VELCRO trademark. The hook element may be provided by a single-prong hook configuration, a multiple-prong hook configuration or by a generally continuous, expanded-head configuration, such as provided by a mushroom-head type of hook element. The loop element may be provided by a woven fabric, a nonwoven fabric, a knitted fabric, a perforated or apertured layer, and the like, as well as combinations thereof. The many arrangements and variations of such fastener systems have been collectively referred to as hook-and-loop fasteners. As illustrated, the hook element is located on the fastener tab 36 and the loop element on the patch 50, but the arrangement of the hook element and the loop element could be reversed.

The absorbent structure 32 has a construction that is generally compressible, conformable, non-irritating to the wearer's skin, and capable of absorbing and retaining body exudates. It should be understood that, for purposes of this invention, the absorbent structure 32 comprises several parts that are assembled together. The absorbent core 33 of the absorbent structure 32 may be constructed of any of a number of absorbent materials, as are well known in the art. For example, the absorbent core 33 may be provided by a layer of coform, meltblown fibers, bonded carded webs, a wetlaid body, tissue laminates, foams, a surge/air formed composite and the like or combinations thereof. In particular, the absorbent core 33 may be provided as a combination of hydrophilic fibers, and high-absorbency material.

In the illustrated embodiment, the absorbent core 33 is zoned, having a selected zone 35 of higher basis weight (FIG. 2). There may be multiple zones or portions of the absorbent core selected to have particular properties. In the illustrated embodiment the zone 35 is constructed and arranged to provide for additional retention of liquid (as compared to the other regions of the core 33). The zone 35 may be positioned in a location where maximum absorbent capacity is needed. However, the reinforcing member of the present invention (e.g., scrim 40) is believed to reduce the need for zoning of the core, as discussed elsewhere herein.

Various types of wettable, hydrophilic fibrous material can be used to provide the fiber material for the absorbent core 33. Examples of suitable fibers include naturally occurring organic fibers composed of intrinsically wettable material, such as cellulosic fibers including wood pulp fibers which can be curled, crosslinked or otherwise mechanically or chemically modified. Other examples of suitable fibers include synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers; and synthetic fibers composed of a nonwettable thermoplastic polymer, such as polypropylene fibers, which have been hydrophilized by appropriate means. The fibers may be hydrophilized, for example, by treatment with silica, treatment with a material that has a suitable hydrophilic moiety and is not readily removable from the fiber, or by sheathing the nonwettable, hydrophobic fiber with a hydrophilic polymer during or after the formation of the fiber. For the purposes of the present invention, it is contemplated that selected blends of the various types of fibers mentioned above may also be employed.

The high-absorbency material used in the absorbent core 33 may comprise absorbent gelling materials, such as superabsorbent materials. Absorbent gelling materials can be natural, biodegradable, synthetic and modified natural polymers and materials. In addition, the absorbent gelling materials can be inorganic materials, such as silica gels, or organic compounds such as cross-linked polymers. The term “cross-linked” refers to any means for effectively rendering normally water-soluble materials substantially water insoluble but swellable. Such means can include, for example, physical entanglement, crystalline domains, covalent bonds, ionic complexes and associations, hydrophilic associations, such as hydrogen bonding, and hydrophobic associations or Van der Waals forces. Examples of synthetic absorbent gelling material polymers include the alkali metal and ammonium salts of poly(acrylic acid) and poly (methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and the like. Mixtures of natural and wholly or partially synthetic absorbent polymers can also be useful in the present invention. Other suitable absorbent gelling materials are disclosed by Assarsson et al. in U.S. Pat. No. 3,901,236 issued Aug. 26, 1975. Processes for preparing synthetic absorbent gelling polymers are disclosed in U.S. Pat. No. 4,076,663 issued Feb. 28, 1978 to Masuda et al. and U.S. Pat. No. 4,286,082 issued Aug. 25, 1981 to Tsubakimoto et al.

Superabsorbent materials are well known in the art, and are readily available from various suppliers. For example, FAVOR SXM 880 superabsorbent is available from Stockhausen, Inc., a business having offices located in Greensboro, N.C., U.S.A.; and DRYTECH 2035 is available from Dow Chemical Company, a business having offices located in Midland, Mich., U.S.A.

The high-absorbency material used in the absorbent core 33 is generally in the form of discrete particles. The particles can be of any desired shape, for example, spiral or semi-spiral, cubic, rod-like, polyhedral, etc. Shapes having a large greatest dimension/smallest dimension ratio, like needles, flakes, and fibers, are also contemplated for use herein. Conglomerates of particles of absorbent gelling material may also be used in the absorbent core 33. Desired for use are particles having an average size of from about 20 micrometers to about 1 millimeter. “Particle size” as used herein means the weighted average of the smallest dimension of the individual particles.

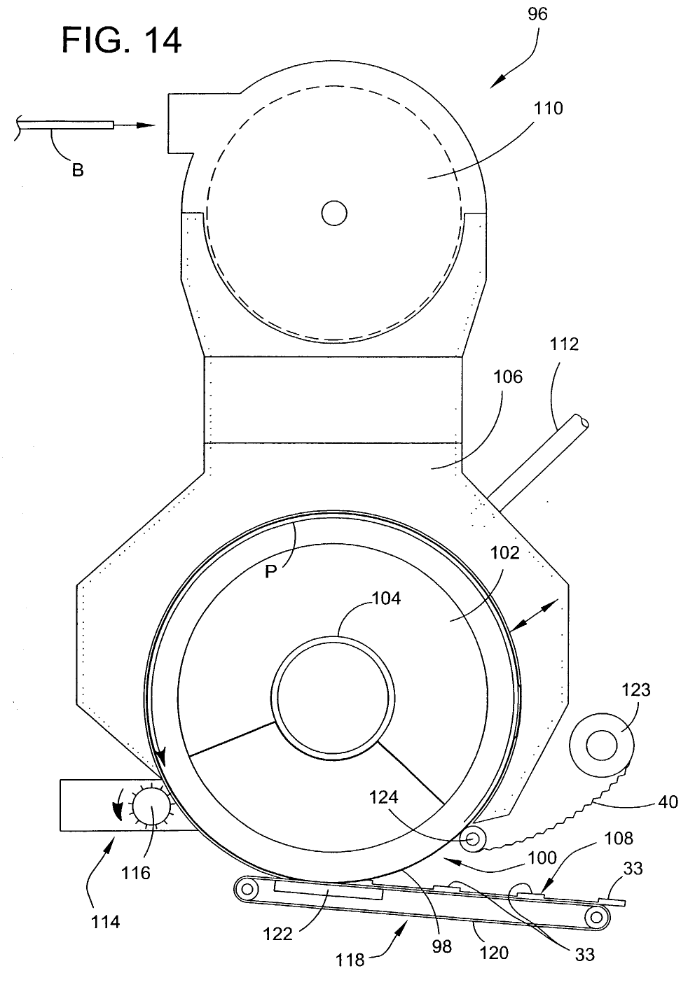

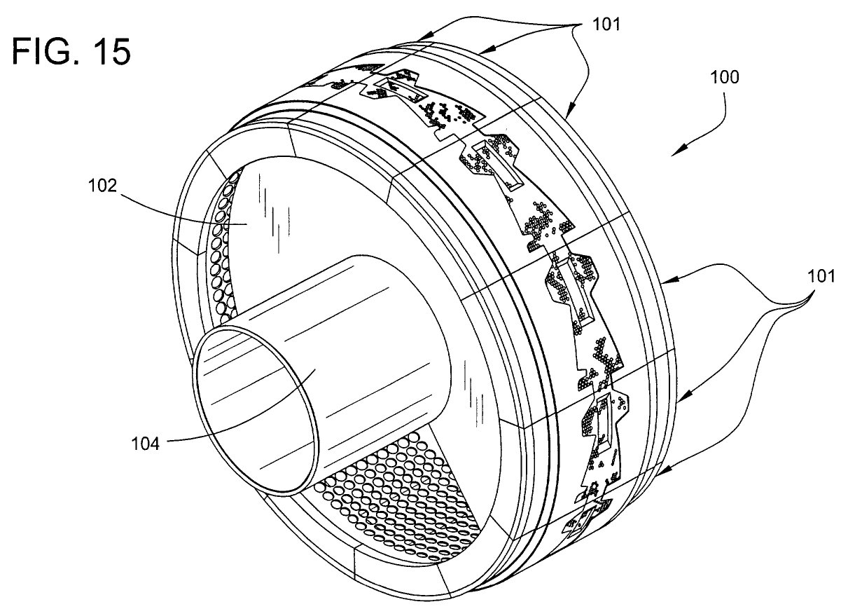

The absorbent materials and superabsorbent materials may be integrated into the absorbent core by employing any operative method or apparatus. For example, the absorbent core may be formed with a dry-forming technique, an air forming technique, a wet-forming technique, a foam-forming technique or the like, as well as combinations thereof. Certain methods and apparatus for carrying out such techniques are well known in the art. One example is described more fully below in reference to FIG. 14.

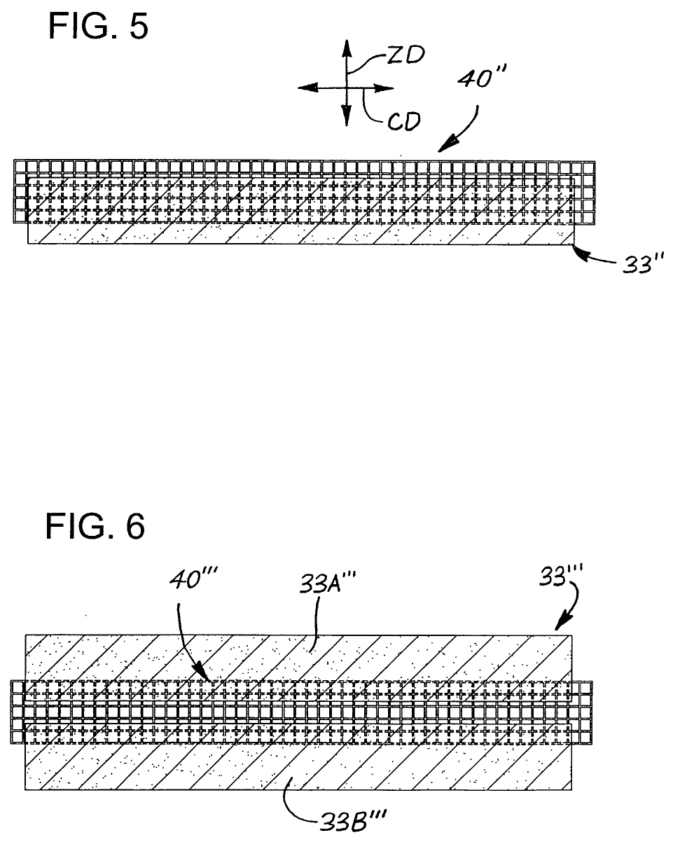

The web of scrim 40 is incorporated into the absorbent core 33 of the absorbent structure 32. In an embodiment illustrated in FIGS. 1-3, the scrim 40 comprises elongate strands 80 which are arranged so that the strands intersect each other. More specifically, the strands 80 are arranged in a grid including parallel strands extending in the machine-direction MD and strands extending in the cross-direction CD defining rectangular openings 82 in the scrim. Among other things, the openings 82 permit liquid in the absorbent core 33 to flow substantially unhindered through the scrim 40. The strands 80 are secured to each other where they intersect to create a lattice providing strength and stability to the absorbent core. In one embodiment, the width of the scrim 40 is equal to the minimum width of the absorbent core 33 (usually located at the portion of the core which is worn through the crotch). In other embodiments, the width of the scrim 40 is between 25% and 100% and more preferably between 50% and 100% of the narrowest width dimension of the absorbent core 33. However, it is also envisioned that the scrim could be wider than the absorbent core. An example is shown in FIG. 5, in which the scrim 40″ is wider than the maximum width of the core 33″. In general, a reinforcing member might have a width which ranges from 25% to 150% of the maximum width of the absorbent core.

The scrim 40 can be made of any suitable material that provides desired levels of strength and flexibility. For example, the strands 80 of the scrim 40 may be composed of natural or synthetic materials, as well as combinations thereof. In a particular arrangement, the material of the strands 80 may include a synthetic polymer (e.g., polyester, polyethylene, polypropylene, nylon, rayon). The synthetic polymer may be monofilament, bicomponent or multicomponent. One conventional way to form scrim of such material is to extrude and orient strands to form a net configuration. Another way of forming such material is by a photomasking process. In that process, a photosensitive resin is deposited on a woven fabric. A mask is applied in the form of the scrim and electromagnetic radiation is used to cure the unmasked portions of the resin. The mask is then removed and the uncured portions of the resin are washed away, leaving the scrim-patterned, cured resin. Natural materials that could be used to form the scrim include cotton, jute, hemp, wool. Alternate materials include glass, carbon and metallic fibers. The reinforcing scrim 40 can be a woven or nonwoven material. The scrim strands in the machine-direction MD and cross-direction CD could be of different materials. In embodiments where there are z-direction ZD extending strands, the material of these may be different from those of strands extending in the machine-direction MD and/or cross-direction CD. Alternately different materials could be used in alternating scrim strands extending in the machine-direction MD, cross-direction CD, and/or z-direction ZD. Moreover, it will be understood that the strands may extend in different directions, such diagonally or along a curve without departing from the scope of the present invention. In one embodiment, the strands 80 may be formed of superabsorbent material. In that event, the scrim 40 would serve a liquid retention function in addition to its reinforcing function. Still further, the scrim 40 could be formed of one material and coated with another material, or be a biodegradable material, such as polylactic acid. An example of a superabsorbent coating is given in co-assigned application Ser. No. 10/246,811 entitled ABSORBENT ARTICLES HAVING A SUPERABSORBENT RETENTION WEB by Newbill et al., filed Sep. 18, 2002 (attorney docket No. 16,739), the disclosure of which is incorporated herein by reference.

The position in the z-direction ZD of the scrim 40 within the absorbent core may be selectively changed. The scrim 40 is shown extending the full length of the absorbent core 33, but may have a lesser or greater length without departing from the scope of the present invention. The absorbent core 33 has longitudinal edges 84. The scrim 40 is narrower than the absorbent core 33 and arranged so that its longitudinal edges 86 are everywhere located inward of the longitudinal edges 84. In another embodiment, as previously stated, the scrim could be wider than a maximum width of an absorbent core. In the first embodiment, longitudinal edges 86 of the scrim 40 are embedded in and shielded by the fibrous material of the absorbent core 33 so that they do not irritate the skin or abrade or poke holes in other parts of the diaper 10. It is noted that the core 33 is partially broken away in FIG. 1, but extends continuously over its length and embeds the scrim 40. It has been found that the scrim 40 may help the absorbent core 33 hold its shape in conformance with the wearer's body thereby improving fit and increasing comfort. However, it is to be understood that scrim (not shown) which extends beyond one or both of the longitudinal edges and or lateral edges of the absorbent core or structure may also be used.

The scrim 40 may be incorporated in the absorbent core 33 in a suitable manner, such as during the formation of the absorbent core. Suitable air forming methods and apparatus for such incorporation are disclosed in co-assigned U.S. patent application Ser. No. ______, entitled PROCESS AND APPARATUS FOR MAKING A REINFORCED FIBROUS ABSORBENT MEMBER by Venturino et al. (attorney docket No. 16,708) and Ser. No. ______, entitled PROCESS AND APPARATUS FOR AIR FORMING AN ARTICLE HAVING A PLURALITY OF REINFORCED SUPERIMPOSED FIBROUS LAYERS by Heyn et al. (attorney docket No. 16,644), and Ser. No. ______, entitled CONTROLLED PLACEMENT OF A REINFORCING WEB WITHIN A FIBROUS ABSORBENT by Venturino et al.(attorney docket No. 18,613), filed simultaneously herewith, the disclosures of which are incorporated herein by reference. It is noted that these forming methods and apparatus promote the entanglement of the fibers with the scrim 40 and with each other during manufacture of the absorbent core 33. However, post-formation entanglement such as by needle punching or hydroentangling may be used to augment the connection. It is also believed that entanglement is augmented by passing the fibrous web of material containing the scrim through a nip or other debulking device, as described below. Further information regarding entanglement of the fibers with the scrim 40 is given in co-assigned U.S. application Ser. No. ______, entitled ABSORBENT ARTICLE WITH REINFORCED ABSORBENT STRUCTURE, by Venturino et al., (attorney docket no. 16,836) filed simultaneously herewith. The disclosure of this application is incorporated herein by reference.

At least some fibers in the core 33 on one side of the scrim 40 pass through the openings 82 in the scrim and are entangled with fibers of the core on an opposite side of the scrim. In addition, at least some fibers from one side of the scrim 40 and at least some fibers from the opposite side are entangled with the strands 80 of the scrim 40 itself so that mechanical connection is also made with the scrim. In this way, there is a strong joining of the fibers of the core and the scrim 40 so that the scrim can reinforce the absorbent core 33 substantially free of any adhesive, fusion or other connection to the absorbent core other than at least one of: entanglement of the fibers with the scrim; entanglement of fibers with fibers entangled with the scrim; and entanglement of fibers with each other where at least one of the fibers passes through the scrim. However, the scrim 40 may be connected by adhesive, fusion or by means other than or in addition to fiber entanglement without departing from the scope of the present invention.

It is recognized that certain processing steps, e.g., debulking, may producing some additional connection between the scrim 40 and fibers of the absorbent core 33, such as by way of hydrogen bonding. For purposes of the present description, such connections do not detract from the connection of the scrim 40 with the fibers of the absorbent core 33 being substantially free of connection other than through entanglement. The absorbent structure of the present invention, at least in one embodiment, does not require the use of an adhesive to bond the scrim 40 with the fibers of the core 33 and does not require fusion of the scrim with the fibers to produce a robust and durable absorbent core.

In use, the scrim 40 holds the matrix of the fibrous material in the absorbent core 33 together against loads applied through movement of the wearer and by liquid in the absorbent core after receiving one or more insults. These loads tend to cause the fibrous material (and hence the absorbent core 33) to tear apart. The scrim 40 resists forces applied to the absorbent core 33 such as but not limited to tensile, compressive, and shear. The scrim 40 allows the absorbent core 33 to have a lower basis weight of fibrous material because of the additional strength. Accordingly, the construction of a thinner absorbent core 33 and a thinner absorbent structure 32 is facilitated.

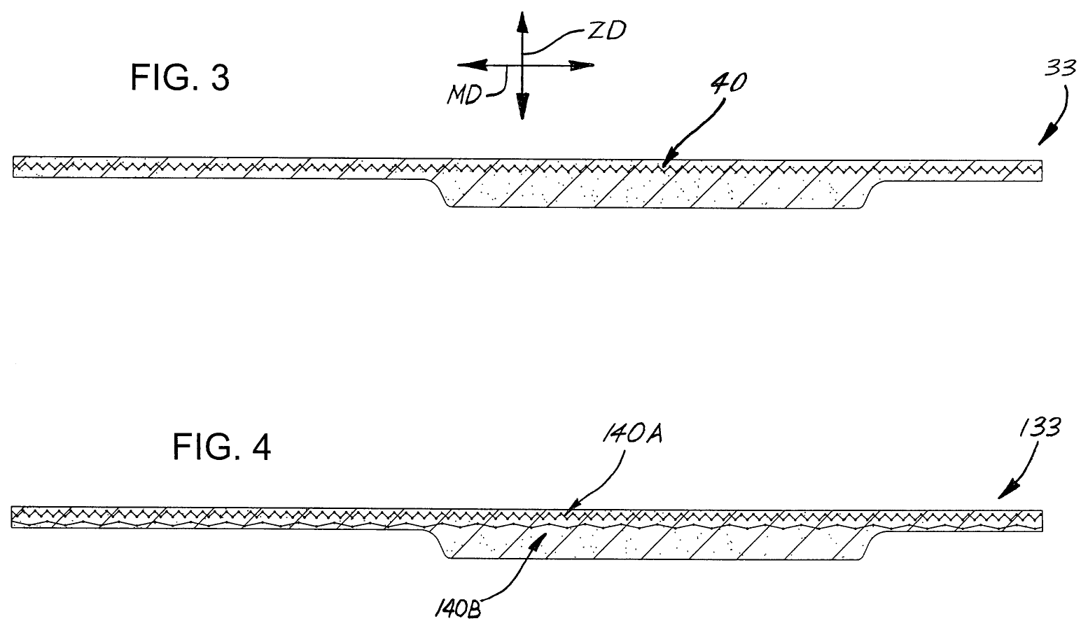

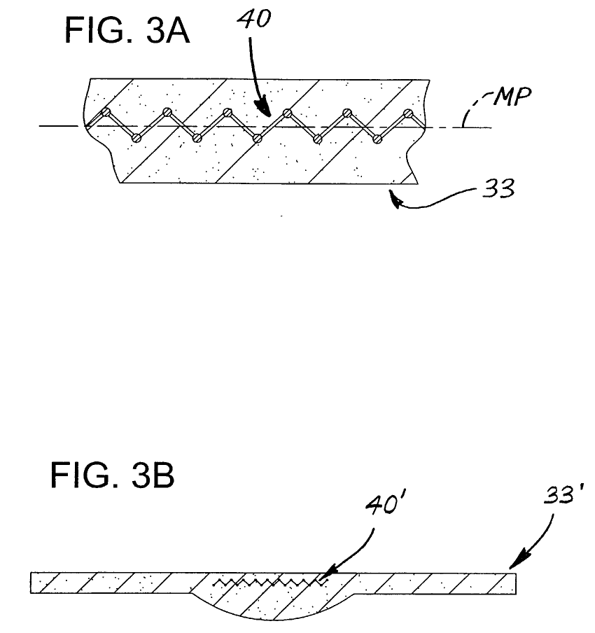

In the illustrated first embodiment, the scrim 40 has a generally accordion shape along its entire length. It is envisioned that the accordion shape could be over less than the entire length and/or in several spaced apart segments, without departing from the scope of the present invention. Moreover as shown in FIG. 3B, the scrim 40′ may be shaped and arranged so that the folds of the accordion extend lengthwise of the absorbent core 33′, rather than widthwise.

Referring to FIG. 3, the scrim 40 is constructed and arranged to resist compaction of the fibers and particles of the absorbent core 33 at least in the thickness or z-direction ZD of the core. The scrim 40 may also act to hold the shape of the core 33 in the machine-direction MD and/or cross direction CD, or in some combination of the three directions MD, CD, ZD. Avoiding compaction of the fibers in the core 33, maintains the void volume of the core, that is, the volume defined by the sum of interstitial volumes defined between fibers within the core. Forces tending to cause compaction and loss of void volume are encountered, for example, when the wearer of the diaper 10 moves. Wetting of the absorbent core 33 also applies loads which tend to compress the core in the z-direction ZD and compact the fibers, reducing permeability of the core to liquid and its original liquid capacity. The construction of the scrim 40, including its material and accordion shape, acts to resist deformation of the core 33 in the z-direction ZD. However that resistance may be overcome, allowing compression of the core 33 to occur. The construction of the scrim 40 allows it to act as a spring to substantially restore the core 33 to its full thickness when the force causing the compression is relieved.

Each fold of the accordion scrim 40 defines a transversely extending rib which will resist compression in the z-direction ZD much more readily than scrim (not shown) which is arranged to lie in a plane or smooth surface. Stated another way, when the absorbent core 33 is laid flat (as shown in FIG. 3), the reinforcing member (e.g., scrim 40) defines a median plane MP (FIG. 3A). The scrim 40 has portions which lie on both sides of the median plane MP. Moving lengthwise along the core 33 on either side of the median plane MP it will be seen that different portions of the scrim 40 lie at different distances from the median plane. In other words, the scrim 40 extends toward and away from the median plane MP over its length. FIG. 3A shows an enlarged fragment of the section of FIG. 3 for clarity in showing the median plane MP. More broadly, the median plane MP is a median surface (i.e., not necessarily planar). The median surface could depart from being planar if the scrim were shaped in a non-uniform manner, or if the scrim was curved over its length or width.

Turning the scrim toward a direction closer to parallel to the thickness of the absorbent core 33 allows the scrim to resist the deformation of the thickness of the core at least partially in compression, rather than in bending. Reduction in the thickness of the absorbent core 33 and accordion scrim 40 will occur, such as during manufacture and when a child wearing the diaper 10 wets, or sits or lies on the absorbent core. However, the scrim 40 is relatively more resilient than the surrounding fibers, and less subject to compression set. Although the accordion scrim 40 deforms, it has a far greater tendency to resume its original shape once the force of compression is relieved. The intimate connection of the scrim 40 with the fibers causes the absorbent core 33 to substantially resume its original shape once the compression force is relieved. As a result, the fibers and particles (at least in the region of the scrim 40) are less compacted, allowing the absorbent core 33 to remain more permeable to liquid and to retain a higher capacity for liquid absorption. By acting to preserve the void volume and liquid permeability of the absorbent core 33, the need to zone the core for additional liquid intake and storage may be reduced or avoided.

A reinforcing member according to the present invention may take on forms other than scrim 40 shown in FIGS. 1-3. For instance, the openings 82 need not be rectangular or regular in size and shape. Moreover, a suitable reinforcing member need not be formed from strands 80. For instance, the reinforcing member could be a shaped film sheet or sheets (not shown). It is desirable for the reinforcing member to be suited for attachment to the fibrous matrix of the absorbent core 33 by fiber entanglement, meaning among other things, that the fibers should be able to wrap around the structure of reinforcing member. However, a reinforcing member could be attached to fibers in the absorbent core other than by entanglement without departing from the scope of the present invention.

In another suitable arrangement shown in FIG. 4, the reinforcing member includes a first scrim element 140A and a second scrim element 140B arranged in layers within the absorbent core 133. In this instance a median surface (not shown) of the reinforcing member might lie between the two elements. However, it will still be understood that the reinforcing member, composed of both of the elements 140A, 140B, extends on both sides of the median surface and is at different distances from the median surface. As shown in FIG. 4, the first element 140A is above the second element 140B. Both elements 140A, 140B have a generally accordion shape, but the second scrim element 140B has fewer folds and correspondingly fewer ribs (i.e., the folds are at a lesser frequency, while their amplitudes are approximately the same as those of the element 140A). Of course, the elements 140A, 140B could have the same shape without departing from the scope of the present invention. It is not necessary for both or either of the elements 140A, 140B to have an irregular shape. For instance, one or both of the elements could have a sinusoidal configuration. It is not necessary that the elements overlie one another, or that they extend continuously over the length of the absorbent core 133. More than two elements could be used. The difference in shape allows for, among other things, some selective variation in the resistance and response to the application of compressive forces. For instance by offsetting the folds of the two elements 140A, 140B, additional resistance to compression (and a greater resiliency) can be achieved. The use of two (or more) elements 140A, 140B allows more reinforcing material to be put into the core 133 inexpensively and without requiring a more complex structure of the reinforcing member. Still further, the two elements (or their component parts) could be made of different materials.

In another arrangement shown in FIG. 5, the reinforcing member takes the form of a scrim 40″ having an accordion shape. The accordion scrim 40″ is partially embedded in an absorbent core 33″. The scrim 40″ projects outwardly from the absorbent core 33″ and may provide a cushion barrier for the core on the side from which the scrim projects. In a still further arrangement shown in FIG. 6, an absorbent core 33′″ includes a first portion 33A′″ and a second portion 33B′″. Accordion-shaped scrim 40′″ is embedded partially in both the first portion 33A′″ and the second portion 33B′″. The scrim 40′″ spaces the first and second portions 33A′″, 33B′″ from each other. The scrim 40′″ also serves to at least partially reinforce each of the portions 33A′″, 33B′″.

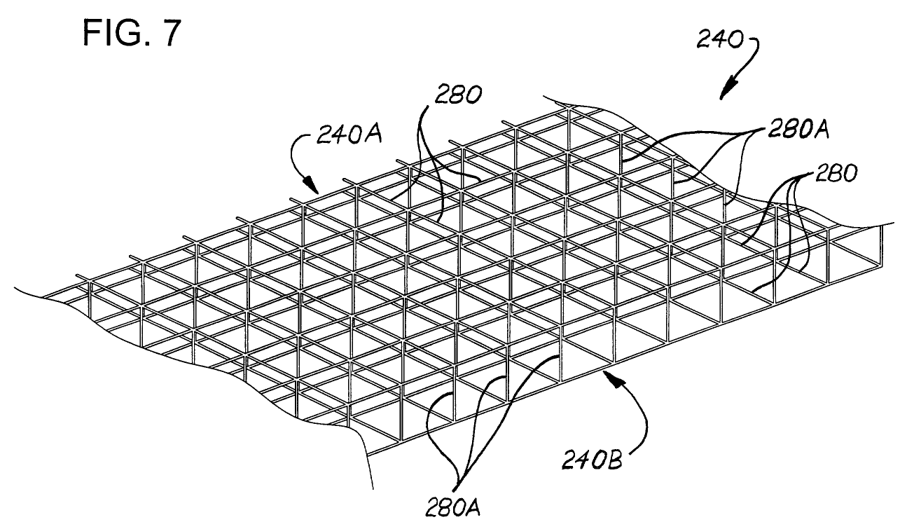

FIG. 7 illustrates another form of the reinforcing member having the shape of a three dimensional or “box” lattice 240. The box lattice includes an upper layer (or element) 240A of generally flat scrim, a lower layer (or element) 240B of generally flat scrim and layer-connecting strands (or cross members) 280A extending between and interconnecting the upper and lower layers. It will be appreciated that this configuration creates a multiplicity of interconnected box beams which will resiliently resist compaction of the fibers and particles of the core (not shown in FIG. 7), by virtue of the strands 280A being essentially parallel to the thickness of the core. The box lattice 240 may resist such compression, but also acts to substantially restore the original thickness once a compressive force is relieved. The same benefits of resistance to bunching, clumping associated with flat scrim are also achieved through the entanglement of the fibers in the core with strands 280, 280A of the box lattice 240.

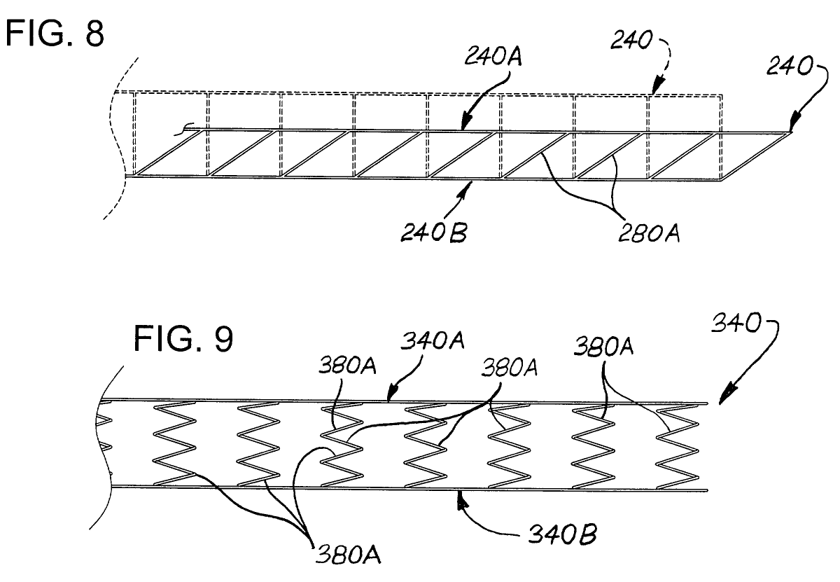

The box lattice 240 may preferably be collapsed prior to use, such as for transporting in a roll (not shown). The solid line illustration of FIG. 8 is the box lattice 240 of FIG. 7 in a collapsed position. The box lattice folds down or “parallelograms” in a regular fashion so that the lattice is not crimped or otherwise damaged in the roll. Once the box lattice web is fed out from the roll, its natural resiliency causes it to pop up to the configuration shown in phantom in FIG. 8. However, it will be understood that the box lattice 240 could naturally assume a collapsed position (e.g., as shown in FIG. 8), but be erected by application of tension to the box lattice web. Once integrated with the fibrous material to in the absorbent core, the box lattice 240 would be held in the erect position and still capable of resiliently resisting thickness compression.

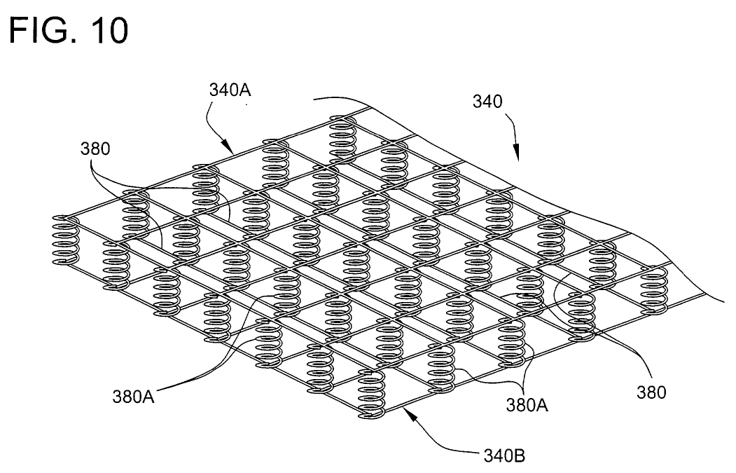

FIGS. 9 and 10 illustrate reinforcing member similar to the box lattice 240 of FIGS. 7 and 8. However, the box spring 340 has helically shaped layer-connecting strands 380A extending between and interconnecting an upper layer 340A and a lower layer 340B. The helical shape of the strands 380A allows the strands to function like a compression spring and is less demanding of the material properties of the strands to produce the desired resiliency.

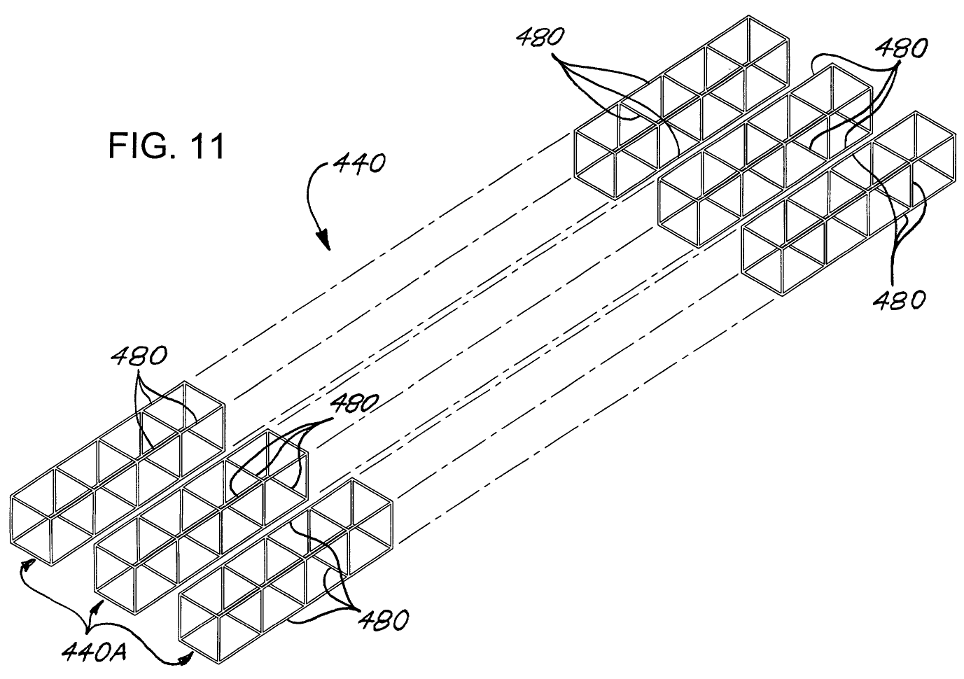

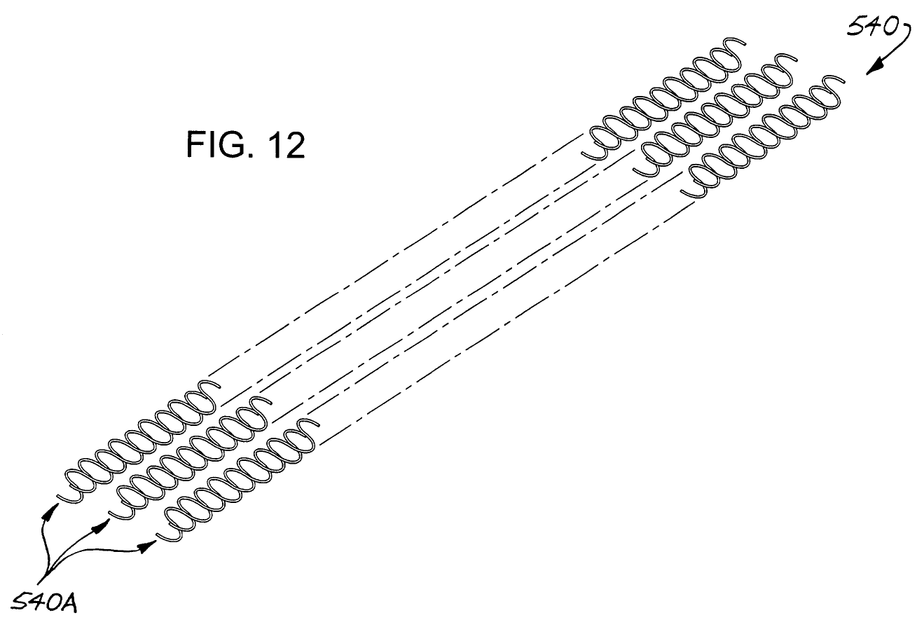

The reinforcing member may take the form of several components which are unconnected to each other except through their common connection to the absorbent core. For instance, the reinforcing member could be two or more separate webs or pieces of scrim (not shown). In one example, laterally spaced pieces of scrim may extend continuously lengthwise of the absorbent core. Other types of reinforcing members including discontinuous components are illustrated in FIGS. 11-13. Referring to FIG. 11, the reinforcing member 440 may take the form of one or more spaced apart square tubes 440A which are unconnected to each other in the absorbent core (not shown in FIG. 11) except through connection with the fibrous matrix of the core. The square tubes 440A are made of strands 480 of polymeric material having a rectangular parallelepiped configuration. In this version, strands 480 extending in one direction (e.g., the vertically extending strands in FIG. 11) may be said to be “cross members” connecting scrim “elements” lying generally in parallel planes. The absorbent core is not illustrated in FIGS. 11-13 for clarity in showing the reinforcing members. The unconnected reinforcing member components have a reduced resistance to tearing forces in directions lateral of the absorbent core, but are constructed to resist longitudinal tearing forces as well as forces tending to compress the thickness of the core. However, the reinforcing member components could extend at any direction within the absorbent core, including in the cross-direction CD and in directions which would intersect each other. Changing the direction of the reinforcing member components would change the types of forces they resist.

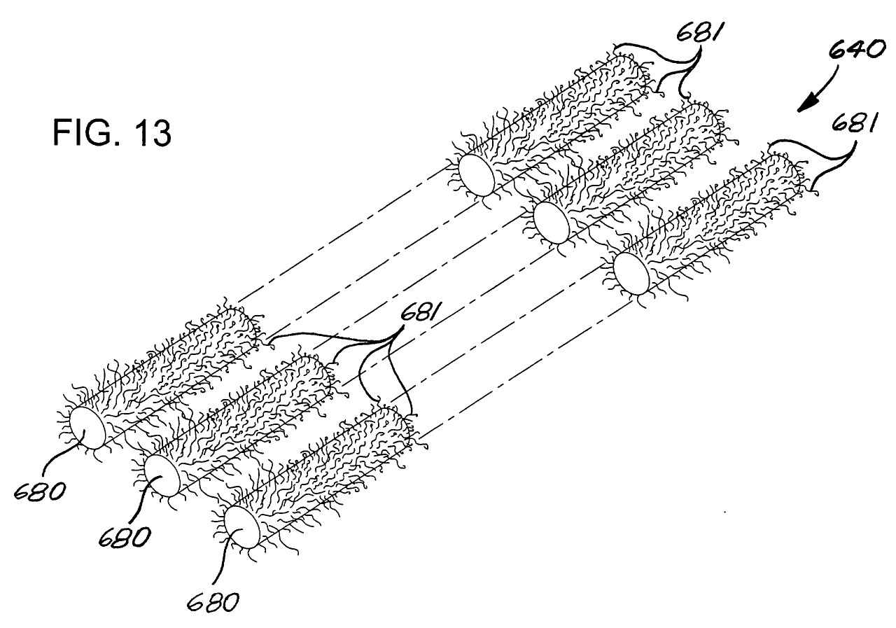

One alternative to the square tubes 440A is a reinforcing member 540 comprising double helixes 540A. Although lacking the box beam structure of the square tubes, the double helixes are able to resiliently resist compressive forces applied perpendicular to their longitudinal axes. Thus, they are able to assist in returning the absorbent core to its uncompressed configuration when the forces tending to compress the thickness of the core are relieved. Another reinforcing member 640 having separate components is shown in FIG. 13. The components are strands 680 which have filaments 681 projecting out from the strands all around them. Thus, the filaments project out from the strands 680 in three dimensions. The fibers of the core are entangled with the filaments 681 to produce the interconnection of the strands 680 and the core. The filaments 681 may provide resistance to compaction of fibers in the absorbent core and resiliency to the core.

In one embodiment, absorbent cores having reinforcing members (40, 140, . . . , 640) may be made using conventional air forming apparatus, such as the type indicated generally at 96 in FIG. 14. The apparatus 96 comprises a movable, foraminous forming surface 98 extending about the circumference of a drum (generally indicated at 100) mounted for rotation about its axis. A vacuum duct 102 located radially inward of the forming surface 98 extends over an arc of an interior diameter of the drum 100 and is arranged for drawing a vacuum under the foraminous forming surface. The vacuum duct 102 is mounted on and in fluid communication with a vacuum conduit 104 connected to a vacuum source (not shown).

The apparatus 96 further comprises a forming chamber 106 through which the forming surface 98 is movable conjointly with the drum 100 upon rotation thereof. The forming chamber 106 is configured in a conventional manner to define an interior volume to which the forming surface 98 is exposed upon movement of the forming surface through the forming chamber. More particularly, in the illustrated embodiment the forming surface 98 moves in a counter-clockwise direction along an arcuate path P within the forming chamber 106 generally from an entrance through which the forming surface enters the forming chamber substantially free of fibrous material, and an exit through which the forming surface exits the forming chamber with a web 108 of absorbent material formed thereon. A reinforcing member (e.g., scrim 40) is fed by a suitable delivery device (e.g., including storage roll 123 to the forming surface 98). The scrim 40 is fed in a web to the forming surface 98 prior to passing into the forming chamber 106. The web of scrim 40 passes over a roller 124 directing the web onto the forming surface. Absorbent cores (e.g., cores 33, 133, etc.) are formed by cutting the absorbent web 108 into appropriately sized lengths.

A conventional source of fibrous material, such as a fiber supply reservoir (not shown) or a fiberizer 110 delivers a fluent fibrous material (e.g., a flow of discrete fibers) into the forming chamber 106. The fiberizer 110 shown in FIG. 14 is operatively positioned above the forming chamber 106 and can be a rotary hammer mill or a rotatable picker roll. However, it is to be understood that the fiberizer 110 may instead be located remote from the forming chamber 106 and that fluent fibrous material may be delivered to the interior of the forming chamber in other ways by other suitable devices and remain within the scope of the present invention. As an example, suitable fiberizers are available from Paper Converting Machine Company, a business having offices located in Green Bay, Wis., U.S.A.

The fibrous material may include natural fibers, synthetic fibers and combinations thereof. Examples of natural fibers include cellulosic fibers (e.g., wood pulp fibers), cotton fibers, wool fibers, silk fibers and the like, as well as combinations thereof. Synthetic fibers can include rayon fibers, polyolefin fibers, polyester fibers and the like, and combinations thereof. The fibrous material employed in the apparatus 96 of FIG. 14 is derived from a batt B of wood pulp cellulose fibers fed to the fiberizer 110 which converts the batt into discrete fibers and delivers fluidized fibrous material into the forming chamber 106.

Other fibrous or particulate material for forming the absorbent web 108 may additionally be delivered into the forming chamber 106. For example, particles or fibers of superabsorbent material may be introduced into the forming chamber 106 by employing conventional mechanisms such as pipes, channels, spreaders, nozzles and the like, as well as combinations thereof. In the illustrated embodiment, superabsorbent material is delivered into the forming chamber 106 by delivery conduit and nozzle system (which is shown schematically in FIG. 14 and indicated at 112). The fibers, particles and other desired material may be entrained in any suitable fluid medium within the forming chamber. Accordingly, any reference herein to air as being the entraining medium should be understood to be a general reference which encompasses any other operative entraining fluid.

The forming chamber 106 is supported by a suitable support frame (not shown) which may be anchored and/or joined to other suitable structural components, as necessary or desirable. The forming surface 98 is illustrated herein as being part of the forming drum 100, but it is to be understood that other techniques for providing the forming surface may also be employed without departing from the scope of the present invention. For example, the forming surface may be provided by an endless forming belt (not shown). A forming belt of this type is shown in U.S. Pat. No. 5,466,409 entitled FORMING BELT FOR THREE-DIMENSIONAL FORMING APPLICATIONS by M. Partridge et al. which issued on Nov. 14, 1995.

In operation, the vacuum source creates a vacuum in the vacuum duct 102 relative to the interior of the forming chamber 106. As the forming surface 98 enters and then moves through the forming chamber 106 along a forming path P toward the exit of the chamber, the fluidized fibrous materials and other particles within the forming chamber are operatively carried or transported by an entraining air stream and drawn inward by the vacuum toward the foraminous forming surface. Air passes inward through the forming surface 98 and is subsequently passed out of the drum 100 through the vacuum duct 102 and vacuum supply conduit 104. Fibers and other particulates are collected by the forming surface 98 as the air passes therethrough such that the collection of fibrous material forms the absorbent web 108 on the forming surface 98.

Subsequently, the forming surface 98 carrying the absorbent web 108 passes out of the forming chamber 106 through the exit to a scarfing system, generally indicated at 114 in FIG. 14, where excess thickness of the absorbent web 108 can be trimmed and removed to a predetermined extent. The scarfing system 114 includes a scarfing roll 116 for abrading excess fibrous material from the absorbent member. The removed fibers are transported away from the scarfing chamber within a suitable discharge conduit (not shown), as is well known in the art.

After the scarfing operation, the portion of the forming surface 98 on which the absorbent web 108 has been formed can be moved to a release zone of the apparatus 96 disposed exteriorly of the forming chamber 106. In the release zone, the absorbent member is drawn away from the forming surface 98 onto a conveyor, which is indicated generally at 118. The release can be assisted by the application of air pressure from the interior of the drum 100. The conveyor 118 receives the formed absorbent web 108 from the forming drum 100, and conveys the absorbent web to a collection area or to a location for further processing (not shown). Suitable conveyors can, for example, include conveyer belts, vacuum drums, transport rollers, electromagnetic suspension conveyors, fluid suspension conveyors or the like, as well as combinations thereof.

In the illustrated embodiment, the conveyor 118 includes an endless conveyor belt 120 disposed about rollers. A vacuum suction box 122 is located below the conveyor belt 120 to draw the absorbent web 108 away from the forming surface 98. The belt 120 is perforated and the vacuum box 122 defines a plenum beneath the portion of the belt in close proximity to the forming surface 98 so that the vacuum within the vacuum box acts on the absorbent web 108 on the forming surface. Removal of the absorbent web 108 from the forming surface 98 can alternatively be accomplished by the weight of the absorbent member, by centrifugal force, by mechanical ejection, by positive air pressure or by some combination thereof or by another suitable method without departing from the scope of this invention. As an example, the removed absorbent web 108 of the illustrated embodiment includes an interconnected series of absorbent cores 33, each of which has a selected surface contour that substantially matches the contours provided by the corresponding portions of the forming surface 98 upon which each individual absorbent core was formed.

The apparatus 96 and method described thus far for air forming a fibrous absorbent member is generally conventional and well known in the art. For example, see U.S. Pat. No. 4,666,647 entitled APPARATUS AND METHOD FOR FORMING A LAID FIBROUS WEB by K. Enloe et al. which issued May 19, 1987; and U.S. Pat. No. 4,761,258 entitled CONTROLLED FORMATION OF LIGHT AND HEAVY FLUFF ZONES by K. Enloe which issued Aug. 2, 1988; the entire disclosures of which are incorporated herein by reference. Other such apparatus are described in U.S. Pat. No. 6,330,735, entitled APPARATUS AND PROCESS FOR FORMING A LAID FIBROUS WEB WITH ENHANCED BASIS WEIGHT CAPABILITY by J. T. Hahn et al. which issued Dec. 18, 2001, and U.S. patent application Ser. No. 09/947,128, entitled MULTI-STAGE FORMING DRUM COMMUTATOR by D. P. Murphy et al., filed Sep. 4, 2001, the entire disclosures of which are incorporated herein by reference. Examples of techniques for introducing a selected quantity of superabsorbent particles into a forming chamber are described in U.S. Pat. No. 4,927,582 entitled METHOD AND APPARATUS FOR CREATING A GRADUATED DISTRIBUTION OF GRANULE MATERIALS IN A FIBER MAT by R. E. Bryson which issued May 22, 1990; the entire disclosure of which is incorporated herein by reference. Therefore, construction and operation of the apparatus 96 will not be further described herein except to the extent necessary to set forth the present invention.

Enhancement of the entanglement of the fibers with the scrim 40 is believed to be further augmented by passing the web through debulking rollers (not shown). The debulking rollers define a nip which is considerably smaller than the thickness of the absorbent web 108 prior to entry into the nip. Thus, the web is compressed and markedly reduced in thickness by operation of the debulking rollers 125. The fibers of the web 108 undergo considerable deformation when passing through the nip of the debulking rollers, especially at high speeds and significant compression. It is believed that the compaction also causes at least some additional fibers to be wrapped around the scrim strands 80, improving entanglement and hydrogen bonding of the absorbent/scrim matrix of the web 108. Moreover, fibers that are already somewhat wrapped around the strands 80 can be further secured to the strands and to the resulting stabilized matrix. In one embodiment, the absorbent core 33 has a density in the range of 0.06 to 0.5 g/cc. It is believed the scrim 40 would have particular advantage in cores having densities in excess of about 0.12 g/cc.

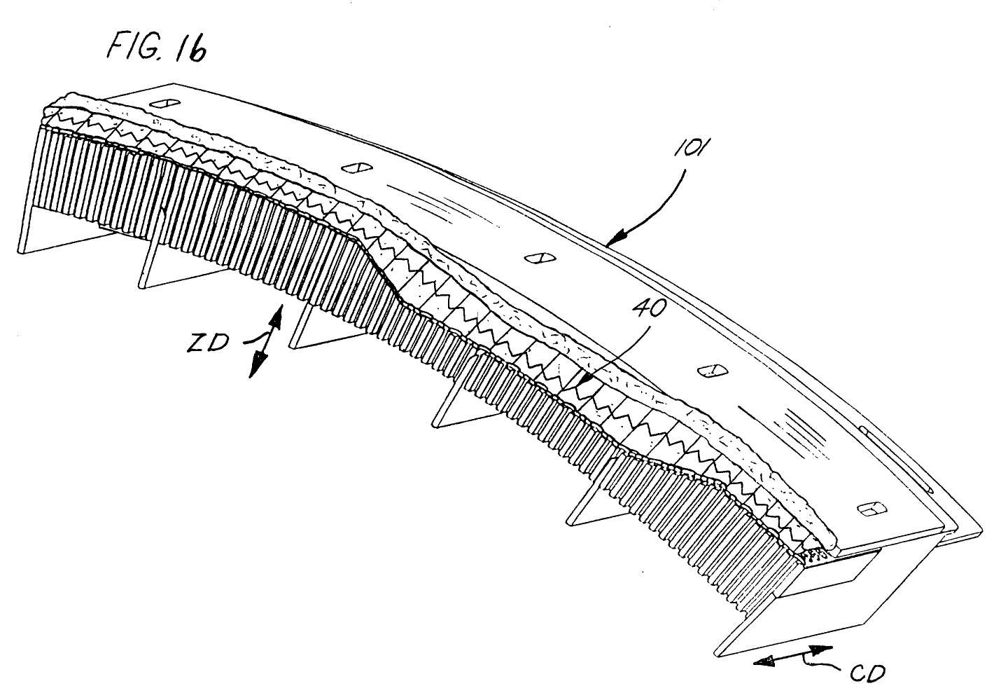

FIG. 15 shows the forming drum 100 apart from the remainder of the apparatus 96. In the illustrated embodiment, the forming drum 100 is made up of a series of form members 101 which are attached to the circumference of the drum. Each of the form members 101 forms a single absorbent core 33. However, as formed the cores 33 are connected together in the unified web 108.

The reinforcing member (40, 140, 240, etc.) can be delivered to the forming surface 98 of the forming apparatus. However as previously discussed herein, the reinforcing member may be in a collapsed (i.e., substantially planar) configuration when on the roll prior to being fed into the forming apparatus. An example of this is shown in FIG. 8 and described above with regard to box lattice 240. However, it is to be understood that other forms of the reinforcing member may be collapsed. For instance, the accordion scrim 40 could lie flat in the roll, but have sufficient memory to pop up into the accordion configuration once fed off of the roll. It is to be understood that it is not necessary for the scrim to have an accordion shape to be capable of being collapsed and erected. For instance the scrim 40 could be made from shape memory polymers or metals and be activated into the three-dimensional shape before or after being incorporated into the absorbent by various mechanisms known in the art (e.g., temperature, humidity, etc.).

To form an absorbent core having a reinforcing member comprising distinct components as shown in FIGS. 11-13, the components may be initially attached together (not shown) and cut just prior to placement onto the forming surface 98. This permits the components to be more easily controlled in manufacture.

To improve the containment of the high-absorbency material, absorbent structure 32 can include an overwrap, such as a wrap sheet 74, which is placed immediately adjacent and around the absorbent core 33 and may be bonded to the absorbent core and to the various other components of the diaper (FIG. 2). The wrap sheet 74 is preferably a layer of absorbent material which covers the major bodyside and outerside surfaces of the absorbent core 33, and preferably encloses substantially all of the peripheral edges of the absorbent core to form a substantially complete envelope thereabout. Alternatively, the wrap sheet 74 can provide an absorbent wrapping which covers the major bodyside and outerside surfaces of the absorbent core 33, and encloses substantially only the lateral side edges of the absorbent core. Accordingly, both the linear and the inwardly curved portions of the lateral side edges of the wrap sheet 74 can be closed about the absorbent core. In such an arrangement, however, the end edges of the wrap sheet 74 may not be completely closed around the end edges of the absorbent core 33 at the waistband regions of the article.

For example, the complete wrap sheet 74, or at least the bodyside layer of the wrap sheet, may comprise a meltblown web composed of meltblown fibers, such as meltblown polypropylene fibers. Another example of the absorbent wrap 74 may comprise a low porosity cellulosic web, such as a tissue composed of an approximately 50/50 blend of hardwood/softwood fibers.

The absorbent wrap 74 may comprise a multi-element wrapsheet which includes a separate bodyside wrap layer and a separate outerside wrap layer, each of which extends past all or some of the peripheral edges of the absorbent core 33. Such a configuration of the wrap sheet can, for example, facilitate the formation of a substantially complete sealing and closure around the peripheral edges of the absorbent core 33. In the back waistband portion of the illustrated diaper 10, the absorbent wrap 74 may also be configured to extend an increased distance away from the periphery of the absorbent core 33 to add opacity and strength to the back side-sections of the diaper 10. In the illustrated embodiment, the bodyside and outerside layers of the absorbent wrap 74 can extend at least about ½ inch beyond the peripheral edges of the absorbent structure to provide an outwardly protruding, flange-type bonding area over which the periphery of the bodyside portion of the absorbent wrap may be completely or partially connected to the periphery of the outerside portion of the absorbent wrap.

The bodyside and outerside layers of the wrap sheet 74 may be composed of substantially the same material, or may be composed of different materials. For example, the outerside layer of the wrap sheet 74 may be composed of a relatively lower basis weight material having a relatively high porosity, such as a wet strength cellulosic tissue composed of softwood pulp. The bodyside layer of the wrap sheet 74 may comprise one of the previously described wrap sheet materials which has a relatively low porosity. The low porosity bodyside layer can better prevent the migration of superabsorbent particles onto the wearer's skin, and the high porosity, lower basis weight outerside layer can help reduce costs.

In desired arrangements, a spacer layer 76 may be interposed between the absorbent structure 32 and the backsheet layer 30 to provide desired benefits (FIG. 2). Where the backsheet layer 30 is vapor permeable, for example, the spacer layer 76 can operatively locate and separate the backsheet layer 30 away from the absorbent structure 32 by a discrete distance. The resultant spacing distance can help to reduce a damp or cool feeling that may arise when the absorbent becomes wetted.

In the various attachments and securements employed in the construction of the article of the invention, it should be readily apparent that any conventional attachment or securement technique may be employed. Such techniques may, for example, include adhesive bonds, cohesive bonds, thermal bonds, sonic bonds, pins, staples, rivets, stitches or the like, as well as combinations thereof.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. An absorbent structure for absorbing liquid comprising an absorbent member being at least partially made of fibers and having a thickness, and a reinforcing member at least partially embedded in the absorbent member for maintaining the structural integrity of the absorbent member, the reinforcing member being constructed and arranged relative to the absorbent member to resist compaction of the absorbent member at least in the thickness direction of the absorbent member.

2. An absorbent structure as set forth in claim 1 wherein the reinforcing member is constructed and arranged relative to the absorbent member for resiliently resisting deformation of the absorbent member at least in the thickness direction.

3. An absorbent structure as set forth in claim 1 wherein the absorbent member has a void volume therein, the reinforcing member being constructed and arranged relative to the absorbent member to inhibit collapse of portions of the void volume.

4. An absorbent structure as set forth in claim 1 wherein the absorbent member is liquid permeable, the reinforcing member being constructed and arranged relative to the absorbent member to maintain liquid permeability of the absorbent structure.

5. An absorbent structure as set forth in claim 1 wherein the reinforcing member has a median surface when the absorbent member is laid flat and the reinforcing member has portions located at different distances from said median surface.

6. An absorbent structure as set forth in claim 2 wherein the reinforcing member has a generally accordion-shaped configuration in longitudinal section of the absorbent structure.

7. An absorbent structure as set forth in claim 2 wherein the reinforcing member has a generally accordion-shaped configuration in transverse section of the absorbent structure.

8. An absorbent structure as set forth in claim 2 wherein the reinforcing member includes a first element and a second element.

9. An absorbent structure as set forth in claim 8 wherein the elements are arranged in layers in the absorbent member.

10. An absorbent structure as set forth in claim 8 wherein the first element is shaped differently from the second element.

11. An absorbent structure as set forth in claim 10 wherein the first and second elements are generally accordion-shaped, but have at least one of a different amplitude and a different frequency.

12. An absorbent structure as set forth in claim 8 wherein the first element and second element are interconnected.

13. An absorbent structure as set forth in claim 12 wherein the first and second elements are interconnected by cross members extending generally therebetween to form a box lattice.

14. An absorbent structure as set forth in claim 13 wherein the cross members extend generally orthogonally to the median surface and first and second elements are generally planar.

15. An absorbent structure as set forth in claim 13 wherein the cross members are generally helically shaped.

16. An absorbent structure as set forth in claim 13 wherein the reinforcing member further comprising third and fourth elements interconnected by cross members to form a box lattice separate from the box lattice formed by the first and second elements.

17. An absorbent structure as set forth in claim 16 wherein the reinforcing member further comprises other elements interconnected by cross members to form at least one other box lattice separate from the box lattices formed by the first and second elements and third and fourth elements.

18. An absorbent structure as set forth in claim 8 wherein the first element and second element are intertwined.

19. An absorbent structure as set forth in claim 18 wherein the first and second elements form a double helix.

20. An absorbent structure as set forth in claim 8 wherein the first and second elements each comprise strands having filaments projecting outwardly from the strands generally around the circumference of the strands.

21. An absorbent structure as set forth in claim 1 wherein the reinforcing member projects outwardly from the absorbent member.

22. An absorbent structure as set forth in claim 21 wherein the absorbent member comprises first and second spaced apart portions, the reinforcing member extending between and being embedded in the first and second portions.

23. An absorbent structure as set forth in claim 1 wherein at least some of the fibers are entangled with the reinforcing member.

24. An absorbent structure as set forth in claim 23 wherein the absorbent member includes at least some fibers entangled with other fibers entangled with the reinforcing member.

25. An absorbent structure as set forth in claim 24 wherein the absorbent member includes fibers from a region of the absorbent member on one side of the reinforcing member and fibers in a region on an opposite side of the absorbent member, at least some fiber from one region being entangled with fibers of the other region through the reinforcing member.

26. An absorbent structure as set forth in claim 1 wherein the reinforcing member is substantially free of attachment to the absorbent member except through at least one of: entanglement of the fibers with the reinforcing member; entanglement of fibers with other fibers entangled with the reinforcing member and entanglement of fibers with each other where at least one of the entangled fibers passes through the reinforcing member.

27. An absorbent structure as set forth in claim 1 wherein the reinforcing member is liquid permeable.

28. An absorbent structure as set forth in claim 1 wherein the fibers comprise absorbent fibers.

29. An absorbent structure as set forth in claim 28 wherein the absorbent fibers comprise cellulosic fibers.

30. An absorbent structure as set forth in claim 1 wherein the fibers further comprise nonabsorbent fibers.

31. An absorbent structure as set forth in claim 1 wherein the fibers comprise nonabsorbent fibers.

32. An absorbent structure as set forth in claim 1 wherein the absorbent member has a minimum width and wherein the reinforcing member has a width which is 25% to 100% of said minimum width of the absorbent member.

33. An absorbent structure as set forth in claim 32 wherein the width of the reinforcing member is 50% to 100% of said minimum width of the absorbent member.

34. An absorbent structure as set forth in claim 1 wherein the absorbent member has a maximum width and wherein the reinforcing member has a width which is 25% to 150% of said maximum width of the absorbent member.

35. An absorbent structure as set forth in claim 1 wherein the absorbent member has at least two zones of different basis weights.

36. An absorbent article comprising a liquid permeable liner, a backsheet layer and an absorbent structure as set forth in claim 1 disposed between the liner and the backsheet layer.

37. An absorbent structure for absorbing liquid comprising an absorbent member being at least partially made of fibers and having a thickness, and a reinforcing member at least partially embedded in the absorbent member for maintaining the structural integrity of the absorbent member, the reinforcing member being attached to fibers from the absorbent member, the reinforcing member having a median surface when the absorbent member is laid flat, the reinforcing member extending on both sides of said median surface.

38. An absorbent structure as set forth in claim 37 wherein the reinforcing member is constructed and arranged relative to the absorbent member for resiliently resisting deformation of the absorbent member at least in the thickness direction.

39. An absorbent structure as set forth in claim 37 wherein the absorbent member has a void volume therein, the reinforcing member being constructed and arranged relative to the absorbent member to inhibit collapse of portions of the void volume.

40. An absorbent structure as set forth in claim 37 wherein the absorbent member is liquid permeable, the reinforcing member being constructed and arranged relative to the absorbent member to maintain liquid permeability of the absorbent structure.

41. An absorbent structure as set forth in claim 37 wherein the reinforcing member has a median surface when the absorbent member is laid flat and the reinforcing member has portions located at different distances from said median surface.

42. An absorbent structure as set forth in claim 38 wherein the reinforcing member has a generally accordion-shaped configuration in longitudinal section of the absorbent structure.

43. An absorbent structure as set forth in claim 38 wherein the reinforcing member has a generally accordion-shaped configuration in transverse section of the absorbent structure.

44. An absorbent structure as set forth in claim 38 wherein the reinforcing member includes a first element and a second element.

45. An absorbent structure as set forth in claim 44 wherein the elements are arranged in layers in the absorbent member.

46. An absorbent structure as set forth in claim 44 wherein the first element is shaped differently from the second element.

47. An absorbent structure as set forth in claim 46 wherein the first and second elements are generally accordion-shaped, but have at least one of a different amplitude and a different frequency.

48. An absorbent structure as set forth in claim 44 wherein the first element and second element are interconnected.

49. An absorbent structure as set forth in claim 48 wherein the first and second elements are interconnected by cross members extending therebetween to form a box lattice.

50. An absorbent structure as set forth in claim 49 wherein the cross members extend generally orthogonally to the median surface and first and second elements are generally planar.

51. An absorbent structure as set forth in claim 49 wherein the cross members are generally helically shaped.

52. An absorbent structure as set forth in claim 49 wherein the reinforcing member further comprising third and fourth elements interconnected by cross members to form a box lattice separate from the box lattice formed by the first and second elements.

53. An absorbent structure as set forth in claim 52 wherein the reinforcing member further comprises other elements interconnected by cross members to form at least one other box lattice separate from the box lattices formed by the first and second elements and third and fourth elements.

54. An absorbent structure as set forth in claim 44 wherein the first element and second element are intertwined.

55. An absorbent structure as set forth in claim 54 wherein the first and second elements form a double helix.

56. An absorbent structure as set forth in claim 44 wherein the first and second elements each comprise strands having filaments projecting outwardly from the strands generally around the circumference of the strands.

57. An absorbent structure as set forth in claim 37 wherein the reinforcing member projects outwardly from the absorbent member.

58. An absorbent structure as set forth in claim 57 wherein the absorbent member comprises first and second spaced apart portions, the reinforcing member extending between and being embedded in the first and second portions.

59. An absorbent article comprising a liquid permeable liner, a backsheet layer and an absorbent structure as set forth in claim 37 disposed between the liner and the backsheet layer.
